(19) United States
(12) Patent Application Publication
(10) Pub. No.: US 2026/0083900 A1
(43) Pub. Date: Mar. 26, 2026

(54) CHEMICAL PUMP HOUSING FOR NEGATIVE PRESSURE SYSTEM

(71) Applicant: Aatru Medical, LLC, Cleveland, OH (US)

(72) Inventor: Thomas E. LASH, Chardon, OH (US)

(21) Appl. No.: 18/895,731

(22) Filed: Sep. 25, 2024

(51) Int. Cl. A61M 1/00; A61F 13/05; F04B 37/10

(52) U.S. Cl. CPC A61M 1/962; A61F 13/05; F04B 37/10

(57) ABSTRACT

A chemical pump assembly and method useful for negative pressure therapy includes a chemical pump housing having an inner chamber and a reactor located within the inner chamber being configured to react with a selected gas found in air so as to consume the selected gas when exposed to air in the inner chamber. An opening is provided in the chemical pump housing that is in fluid communication with the inner chamber. A cover is disposed over at least a portion of the chemical pump housing for sealing the opening to prevent ingress of air through the opening into the inner chamber after activation of the reactor. The cover includes a cover body having an underside surface facing the chemical pump housing. The underside surface has an adhesive thereon for affixing the underside surface to the chemical pump housing a removable liner disposed over the adhesive on the underside surface, the removable liner including a pull tab extending laterally away from the chemical pump housing.

### BACKGROUND

Negative pressure therapy is a therapeutic treatment that utilizes negative pressure for skin treatments and restorative purposes. Negative pressure is a term used to describe a pressure that is below normal atmospheric pressure. Negative pressure therapy is utilized for several sites on the skin, such as a wound or an incision. Furthermore, negative pressure therapy is useful to manage wounds with complex healing concerns. Additionally, negative pressure therapy could also be used for cosmetic purposes like removing wrinkles.

Generally, negative pressure therapy is achieved by maintaining a reduced pressure beneath a dressing on a dressing site. A vacuum generation source, such as a pump, applies reduced pressure to the inside of the dressing on the dressing site.

### SUMMARY

According to one aspect, a chemical pump assembly useful for negative pressure therapy includes a chemical pump housing having an inner chamber and a reactor located within the inner chamber being configured to react with a selected gas found in air so as to consume the selected gas when exposed to air in the inner chamber. An opening is provided in the chemical pump housing that is in fluid communication with the inner chamber. A cover is disposed over at least a portion of the chemical pump housing for sealing the opening to prevent ingress of air through the opening into the inner chamber after activation of the reactor. The cover includes a cover body having an underside surface facing the chemical pump housing. The underside surface has an adhesive thereon for affixing the underside surface to the chemical pump housing a removable liner disposed over the adhesive on the underside surface, the removable liner including a pull tab extending laterally away from the chemical pump housing.

According to another aspect, a method for operating a pump assembly of a negative pressure system, includes providing a fluid connection between a chemical pump housing and a dressing placed over a tissue site. The chemical pump housing has an inner chamber and an opening fluidly connected to the inner chamber. The method further includes activating a reactor located within the inner chamber of the chemical pump housing to enable the reactor to react with a selected gas found in air so as to consume the selected gas when exposed to air in the inner chamber. After activating the reactor, the method also includes covering the opening with a cover disposed over at least a portion of the chemical pump housing for sealing the opening to prevent ingress of air through the opening into the inner chamber. Covering the opening includes removing a removable liner disposed over an adhesive on an underside surface of a cover body by pulling a pull tab of the removable liner that extends laterally away from the chemical pump housing in a lateral direction across the cover body.

According to a further aspect, a cover for a chemical pump assembly useful for negative pressure therapy includes a cover body having an underside surface that faces an associated chemical pump housing. The underside surface has an adhesive thereon for affixing the underside surface to the associated chemical pump housing. A removable liner is disposed over the adhesive on the underside surface, the removable liner including a pull tab extending laterally away from the chemical pump housing.

### BRIEF DESCRIPTION OF THE DRAWINGS

FIG. 1 is a perspective view of a chemical pump assembly connected to a dressing.

FIG. 2 is another perspective view of the chemical pump assembly.

FIG. 3 is an exploded perspective view of the chemical pump assembly.

FIG. 4 is another exploded perspective view of the chemical pump assembly.

FIG. 5 is a perspective view similar to FIG. 2 but showing a pull tab of a removable liner being removed from a cover body of a cover to reveal an adhesive on an underside of the cover body for sealing over an opening provided in a chemical pump housing of the chemical pump assembly.

FIG. 6 is a perspective view of the chemical pump assembly after a pair of pull tabs have been removed.

FIG. 7 is a perspective view of a known chemical pump assembly having pull tabs arranged so as to be pulled in a common direction relative to one another.

### DETAILED DESCRIPTION

The invention is not limited in its application to the details of construction and arrangement of components provided in the following description or illustrated in the attached drawings. The invention is capable of other embodiments and being practiced in various manners. The phraseology and terminology used herein is for the purpose of description and should not be regarded as limiting. Moreover, the use of “including,” “comprising,” or “having” and variations thereof is meant to encompass the items listed thereafter and equivalents thereof as well as additional items.

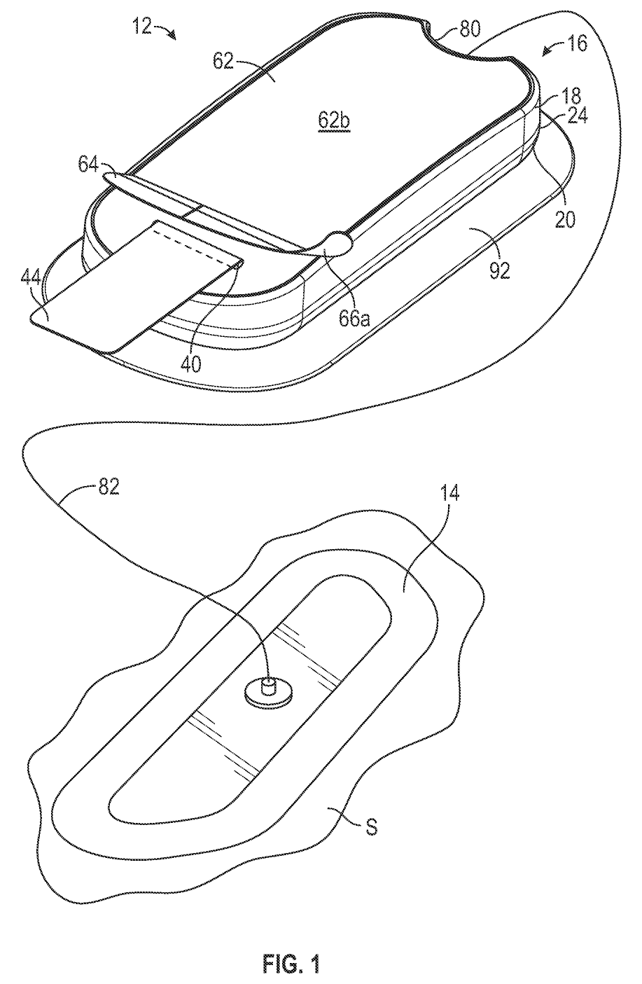

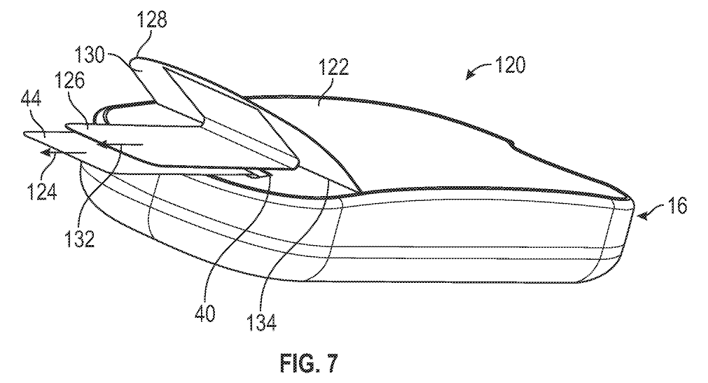

FIG. 7 depicts a known chemical pump assembly 120 useful for negative pressure therapy. An example of this known chemical pump assembly 120 is described in U.S. Patent No. 11,083,833, expressly incorporated herein by reference. Negative pressure described herein is pressure below atmospheric pressure. The chemical pump assembly 120 can be configured to connect with a dressing 14 (see FIG. 1) affixed to skin S so as to be in fluid communication with an enclosed volume beneath the dressing 14. An example of the dressing 14 that can be used with the chemical pump assembly 120 is described in U.S. Patent No. 11,007,083, also expressly incorporated herein by reference.

As shown, the chemical pump assembly 120 includes a cover 122 in the form of a thin film for sealing an opening 40 of a chemical pump housing 16 to prevent ingress of air through the opening 40 into an inner chamber (not shown in FIG. 7) of the chemical pump housing 16 after a reactor (also not shown in FIG. 7) is activated, such as by pulling first pull tab 44. Pulling of the first pull tab 44 occurs by gripping the exposed portion of the pull tab 44 (i.e., the exposed portion being that which is visible in FIG. 7) and pulling the pull tab 44 in the direction of arrow 124. In particular, the pulling direction represented by arrow 124 is generally parallel to a longitudinal length of the pull tab 44 and to a longitudinal length of the chemical pump housing 16.

A second pull tab 126 cooperates with the cover 122, which is placed over and adhered to a portion of the top surface of the chemical pump housing 16. The cover 122 includes a flap 128 with the opening 40 disposed underneath the flap 128. The second pull tab 126 is connected to a release layer 130 provided on a bottom surface of the cover 122. The release layer 130 covers an adhesive (not visible in FIG. 7) on the bottom surface of the cover 122. When the second pull tab 126 is pulled, the second pull tab 126 disconnects the release layer 130 from the flap 128 and the adhesive disposed on the bottom surface of the flap 128 is exposed. The flap 128 can then be moved towards the chemical pump housing 16 to cover the remainder of the top surface of the chemical pump housing 16 and thus also covers the opening 40. In result, the reactor in the chemical pump housing 16 is no longer exposed to ambient atmosphere via the opening 40.

Pulling of the second pull tab 126 occurs by gripping the portion of the second pull tab 126 overlaying the first pull tab 44 and pulling the second pull tab 126 in the direction of arrow 132. In particular, the pulling direction represented by arrow 132 is the same as the pulling direction represented by arrow 124 and thus is generally parallel to a longitudinal length of the pull tab 44 and to a longitudinal length of the chemical pump housing 16. A potential issue can arise if the pulling force applied to the pull tab 44 is too much. In particular, too much pulling force can cause the pull tab 44 to tear or sever from the release layer 130, which leave the release layer 130 still covering the adhesive and now there is no pull tab remaining to remove the release layer 130. It is believed that this is caused in part from the pulling direction (i.e.., the direction of arrow 124) being perpendicular to slit 134 extending laterally across the release layer 130.

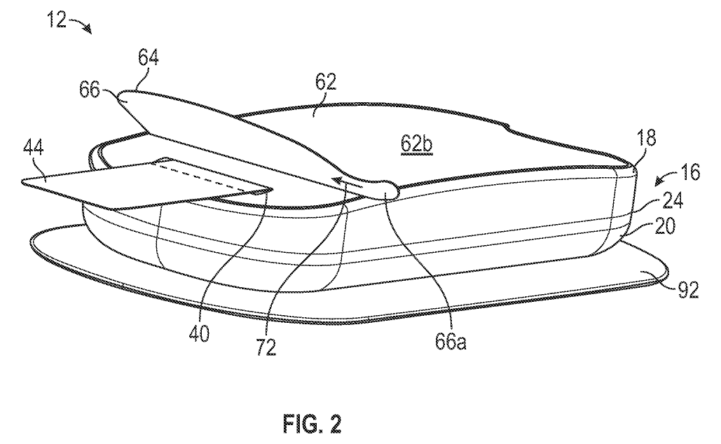

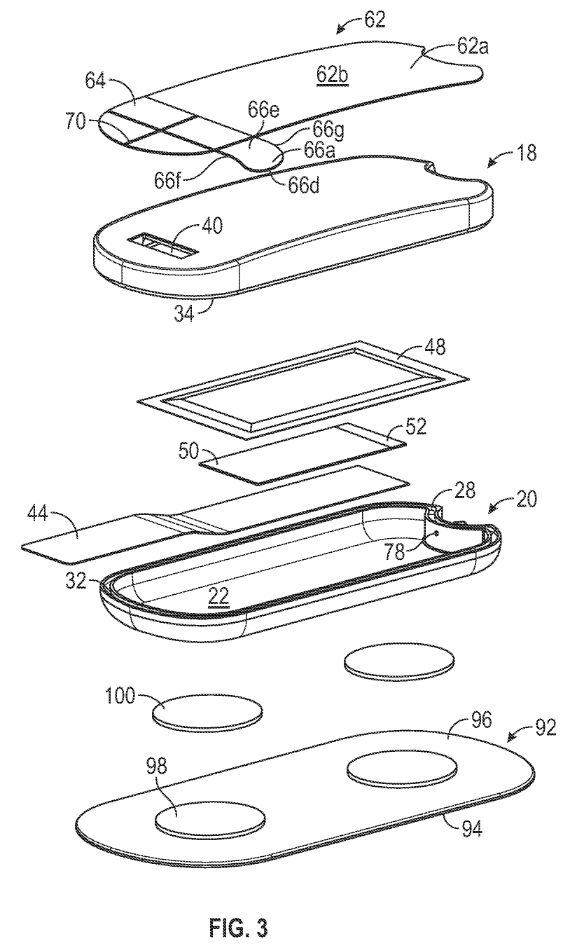

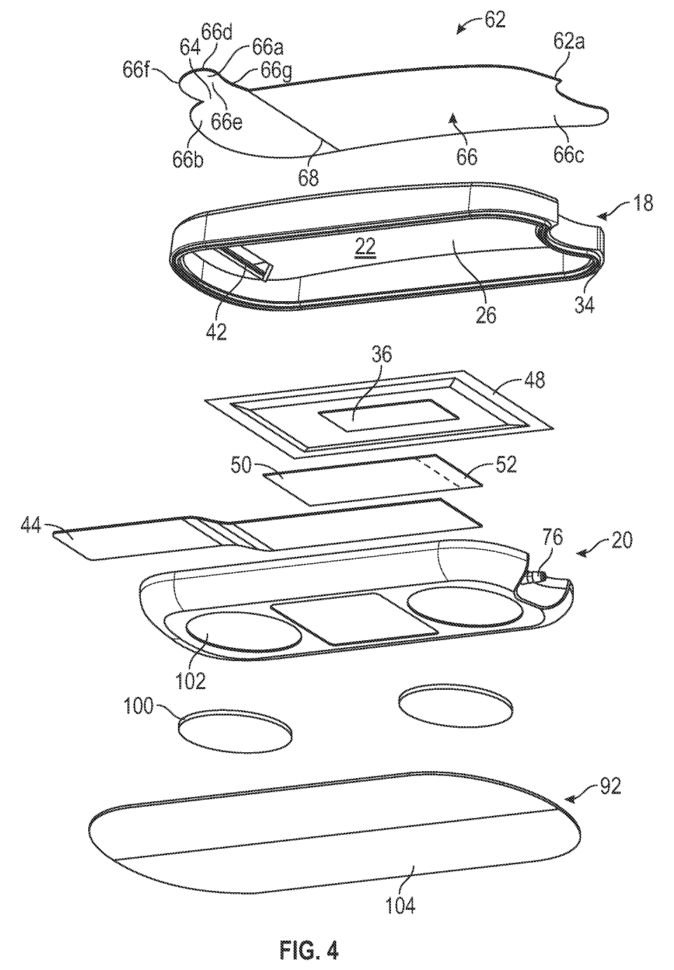

With reference now to FIG. 1, a chemical pump assembly 12 will be described according to one exemplary embodiment of the present disclosure. The chemical pump assembly 12 generally includes pump housing 16, which can be the same pump housing as shown in the known design of FIG. 7. With additional reference to FIGS. 2-4, the chemical pump housing 16 can include an upper housing 18 and a lower housing 20 that connect to define an inner chamber 22 (FIG. 3) disposed there between. In one embodiment, the upper housing 18 and the lower housing 20 can be constructed as separate elements. When the upper housing 18 and the lower housing 20 are separate elements, the upper housing 18 and the lower housing 20 are joined together, and a seam 24 is formed between the upper housing 18 and the lower housing 20. When the upper housing 18 and the lower housing 20 are joined, an air tight seal is formed between the upper housing 18 and the lower housing 20. In result, no gas can enter or escape the inner chamber 22 of the chemical pump housing 16 through the seam 24. In another embodiment, the upper housing 18 and the lower housing 20 could be integrally formed. Furthermore, the upper housing 18 may include an upper inner wall surface 26 which is only slightly curved and nearly planar, as depicted in FIG. 4. The lower housing 20 may include a lower inner wall 28 offset from the sidewalls of the lower housing 20.

The lower housing 20 may further include a channel 32, as shown in FIG. 3, disposed around the inner periphery of the lower housing 20. The channel 32 may surround the entire inner periphery of the lower housing 20 or surround only a portion of the inner periphery of the lower housing 20. Furthermore, the channel 32 can be comprised of a single channel or multiple channels. The channel 32 may be disposed between the sidewalls of the lower housing 20 and the lower inner wall 28. On the upper housing 18, a ridge 34 may surround the inner periphery of the upper housing 18, as shown in FIG. 4. The ridge 34 may surround the entire inner periphery of the upper housing 18 or surround only a portion of the inner periphery of the upper housing 18. The ridge 34 can be comprised of a single ridge or multiple ridges. Alternatively, the ridge 34 may be disposed on the lower housing 20, and the channel 32 may be disposed on the upper housing 18.

The ridge 34 is configured to be inserted into the channel 32 when the upper housing 18 and the lower housing 20 are joined. When the ridge 34 is inserted into the channel 32, the upper housing 18 and the lower housing 20 can be welded and the air tight seal is created to prevent gas from passing through the seam 24. The upper housing 18 and the lower housing 20 can connect in other known manners to provide an air tight seal at the seam 24.

The chemical pump assembly 12 further includes a chemical pump 36. The chemical pump 36 is positioned in the inner chamber 22 of the chemical pump housing 16 prior to connecting the upper housing 18 and the lower housing 20. In the illustrated embodiment, the chemical pump 36 in the chemical pump assembly 12 is a reactor configured to react with a selected gas, e.g., oxygen, found in air. Examples of reactors that can be used in the chemical pump assembly 12 are described in US 2014/0109890A1 and PCT/US2016/059364, both expressly incorporated herein by reference.

Opening 40, which can be in the form of an elongate slit in the illustrated embodiment, is disposed on the upper housing 18. The opening 40 is preferably positioned towards a distal side of the upper housing 18. However, the opening 40 can be positioned towards a proximal section of the upper housing 18 as well as elsewhere on the chemical pump housing 16. When not covered, the opening 40 exposes the inner chamber 22 to ambient atmosphere. Adjacent the opening 40, the upper housing 18 can also include a sloped wall 42 that slopes upwardly and toward the distal side of the upper housing 18 from the inner chamber 22 toward the outer surface. The first pull tab 44 can extend from the inner chamber 22 to ambient through the opening 40.

A packet 48 including a removable layer 50 covers or shields the chemical pump 36 so as to prevent the chemical pump 36 from being exposed to ambient atmosphere or air within the inner chamber 22 until after removal of the removable layer 50 from the packet 48. The packet 48 can be a foil packet that is hermetically sealed around the chemical pump 36. The first pull tab 44 extends through the opening 40 and is connected to removable layer 50. The first pull tab 44 can be pulled to remove the first pull tab 44 from the opening 40. When the first pull tab 44 is pulled through the opening 40, the removable layer 50 is removed from the packet 48 and, if desired, from the inner chamber 22 through the opening 40, exposing the chemical pump 36 to ambient atmosphere. After the removal of the removable layer 50, the chemical pump 36 begins to react with a selected gas, e.g., oxygen, in the inner chamber 22. Since the wall 42 is sloped, the first pull tab 44 and the removable layer 50 are removed from the opening 40 with ease. The first pull tab 44 is preferably removed after the chemical pump assembly 12 is connected to the dressing 14. However, the first pull tab 44 can be removed prior to affixing the chemical pump assembly 12 to the dressing 14.

In the illustrated embodiment, the packet 48 is affixed to the upper inner wall surface 26 of the upper housing 18 through an adhesive. The packet 48 could be affixed to another surface, if desired. The removable layer 50 is coated on an upper side (per the orientation shown in FIG. 3) with an adhesive, with the exception of a small section 52 at and end of the removable layer 50 opposite from the opening 40. The upper side of the removable layer being the side facing the packet 48. The first pull tab 44 connects with this small section 52, which lacks the adhesive, and the connection between the first pull tab 44 and the removable layer 50 is limited to the small section 52 in that the first pull tab 44 is free to move with respect to the remainder of the removable layer 50 that carries the adhesive on the upper side and is affixed to the packet 48. As such, when the first pull tab 44 is pulled away from the chemical pump housing 16 through the opening 40, the removable layer 50 rolls over on itself as the removable layer 50 is peeled away from the packet 48.

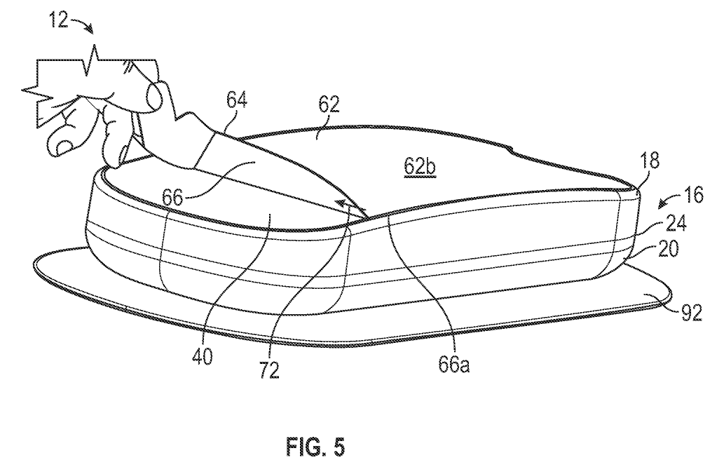

The chemical pump assembly 12 further includes a cover 62 disposed over at least a portion of the chemical pump housing 16 for sealing the opening to prevent ingress of air through the opening into the inner chamber after activation of the reactor 36, such as can occur after the removable layer 50 has been removed. The cover 62 can include a cover body 62a, which can be in the form of a thin film, having a show surface 62b facing away from the chemical pump housing 16 (i.e., the surface facing upward in FIGS. 1-3) and an underside surface 62c facing the chemical pump housing 16 (see FIG. 5). As shown in FIG. 5, the underside surface 62c can have an adhesive 62d thereon for affixing the underside surface 62c to the chemical pump housing 16. Optionally, the cover body 62a can include a flap 64 as will be described in more detail below. Also optionally, the cover 62 can be metallized to prevent the ingress of air into the inner chamber 22 when the inner chamber 22 is at negative pressure and the cover is affixed to the chemical pump housing 16 as will be described in further detail below.

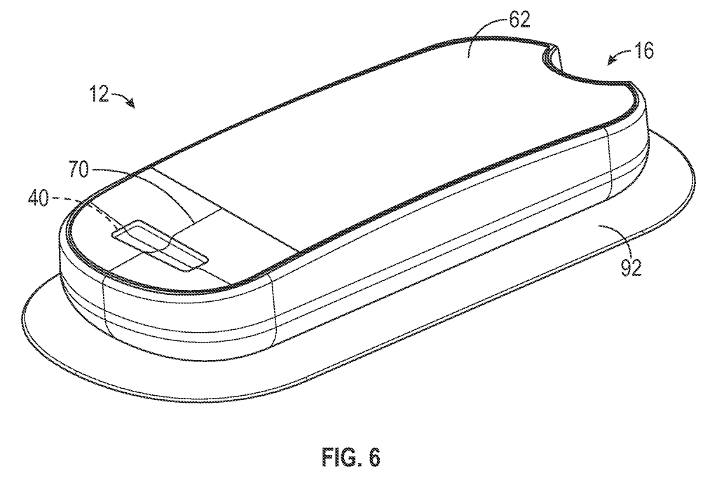

The cover 62 can further include a releasable liner 66, also referred to herein as a removable liner, disposed over the adhesive 62d on the underside surface 62c. The releasable liner 66 can include a removable liner pull tab 66a extending laterally away from the chemical pump housing 16 as shown. In the illustrated embodiment, the releasable liner 66 includes a first section 66b and a second section 66c. The first and second sections 66b, 66c can be independently removable from the cover body 62a relative to one another. In particular, the first section 66b can be disposed over a corresponding first portion of the cover body 62a and the second section 66c can be disposed over a corresponding second portion of the cover body 62. The second section 66c can be removed to expose the adhesive 62d thereunder to allow the second portion of the cover body 62a covered by the second section 66c to be affixed to the pump housing 16 rearwardly of the opening 40 (as shown in FIGS. 1 and 2). The first portion section 66b can be removed to expose the adhesive 62d thereunder to allow the first portion of the cover body 62a covered by the first section 66b, which is positioned to be over and encompassing of the opening 40, to be affixed to the pump housing 16 to close the opening 40 (as shown in FIG. 6). As shown, the first and second sections 66b, 66c define a slit line 68 extending laterally across the cover body 62a at a location where the first and second sections 66b, 66c meet one another. The slit 68 can also define a fold line for the flap 64.

The pull tab 66a can be formed integrally with the first section 66b of the release liner 66. Also, the pull tab 66a can be disposed at the location of the slit line 68. More particularly, the pull tab 66a can include a distal end 66d, a proximal end 66e, a first side 66f and a second side 66g. As shown, the second side 66g can be colinear with the slit line 68 so that removal of the removable liner 66 from the cover body 62a occurs in result of a removable force applied in a direction parallel to the slit line as represented by arrow 72. Notably, this direction is perpendicular to the longitudinal length of the chemical pump housing 16 and of the cover 62. Accordingly, and contrasting with FIG. 7, the direction represented by arrow 72 along which the pull tab 66a is pulled is perpendicular relative to the direction of the arrow 132 along which the pull tab 126 is pulled. Advantageously, this pulling action is parallel or in line with the slit 68, which has the effect of decreasing the likelihood of the pull tab 68a severing or tearing from the release liner 66.

When the pull tab 66a is pulled in the direction of arrow 72, the pull tab 66a disconnects the release layer 66, or at least the first section 66b of the release layer 66, from the flap 64 and the adhesive 62d disposed on the bottom surface of the flap 64 is exposed. The flap 64 can then be moved towards the upper housing 18 to cover the remainder of the top surface of the upper housing 18 and thus also covers the opening 40. In result, the inner chamber 22 is no longer exposed to ambient atmosphere via the opening 40. When the cover 62 covers the opening 40, the chemical pump 36 reacts with the selected gas found in the enclosed volume under the dressing, and if already connected to the dressing via a hose 82 (shown schematically in FIG. 1) creates a closed system. Reduced pressure is therefore developed within the enclosed volume. When the inner chamber 22 is under negative pressure, the cover 62 can be drawn in through the opening 40 toward the inner chamber 22. As such, the cover 62 cooperating with the opening 40 can provide an indication to the user that the inner chamber 22 is under negative pressure. Optionally, indicia 70, e.g. lines, a cross or the like, can also be provided on the cover 62 in the vicinity of the opening 40 to provide further indication of negative pressure.

A method for operating the chemical pump assembly 12 will be described hereinafter. At least one dressing 14 can be placed over a tissue site. The chemical pump assembly 12 can then fluidly connect to the at least one dressing 14 via the hose 82 thereby providing a fluid connection between the chemical pump housing 16 and the dressing 14. When the chemical pump assembly 12 is connected to the at least one dressing 14, the inner chamber 22 of the chemical pump assembly 12 is in fluid communication with the enclosed volume defined by the dressing 14.

Next, the chemical pump 36, which can be a reactor, that is located within the inner chamber 22 of the chemical pump housing 16 can be activated to enable the chemical pump 36 (or reactor) to react with a selected gas found in air so as to consume the selected gas when exposed to air in the inner chamber 22. Such activating can including pulling the pull tab 44 (which can be referred to as a first pull tab), which extends from the inner chamber 22 of the chemical pump housing 16 to ambient through the opening 40 in the chemical pump housing 16, to remove the removable layer 50 to expose the reactor located within the inner chamber 22 to air. More particularly, when the first pull tab 44 is pulled through the opening 40, the removable layer 50 is removed from the packet 48. In result, the chemical pump 36 in the chemical pump housing 16 is exposed to ambient atmosphere as well as air in the inner chamber 22 and begins to react with a selected gas.

After activating the reactor 36, the opening 40 can be covered with the cover 62 disposed over at least a portion of the chemical pump housing 16 for sealing the opening 40 to prevent ingress of air through the opening 40 into the inner chamber 22. This covering of the opening 40 includes removing the removable liner 66, and particularly the first section 66b thereof, disposed over the adhesive 62d on the underside surface 62c of the cover body 62a by pulling the pull tab 66a (which can be referred to as a second pull tab) of the removable liner 66 that extends laterally away for the chemical pump housing 16 in a lateral direction (the direction of arrow 72) across the cover body 62a.

After the adhesive 62d is exposed, the flap 54 can then brought toward the upper housing 18 by pushing the underside surface 62c toward the chemical pump housing 16 to cover the opening 40 with the cover 62. As a result, the inner chamber 22 is no longer exposed to ambient atmosphere. The reactor then reacts with the selected gas in the inner chamber 22 and the enclosed volume beneath the dressing 14 and applies reduced pressure at the tissue site.

It will be appreciated that various of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A chemical pump assembly useful for negative pressure therapy comprising:
a chemical pump housing having an inner chamber;
a reactor located within the inner chamber being configured to react with a selected gas found in air so as to consume the selected gas when exposed to air in the inner chamber;
an opening provided in the chemical pump housing that is in fluid communication with the inner chamber; and
a cover disposed over at least a portion of the chemical pump housing for sealing the opening to prevent ingress of air through the opening into the inner chamber after activation of the reactor, the cover including:
a cover body having an underside surface facing the chemical pump housing, the underside surface having an adhesive thereon for affixing the underside surface to the chemical pump housing, and
a removable liner disposed over the adhesive on the underside surface, the removable liner including a pull tab extending laterally away from the chemical pump housing.

2. The chemical pump assembly of claim 1 wherein the cover body is a thin and/or metallized film.

3. The chemical pump assembly of claim 1 wherein the removable liner includes a first section and a second section, the first and second sections independently removable from the cover body relative to one another, and wherein the pull tab is formed integrally with the first section.

4. The chemical pump assembly of claim 3 wherein the first and second sections define a slit line extending laterally across the cover body at a location where the first and second sections meet one another.

5. The chemical pump assembly of claim 4 wherein the pull tab is disposed at the location of the slit line.

6. The chemical pump assembly of claim 5 wherein the pull tab includes a distal end, a proximal end, a first side and a second side, and wherein the second side is colinear with the slit line so that removal of the removable liner from the cover body occurs in result of a removable force applied in a direction parallel to the slit line.

7. The chemical pump assembly of claim 4 wherein the first section is disposed over a corresponding first portion of the cover body and the second section is disposed over a corresponding second portion of the cover body, the first portion of the cover body positioned to be over and encompassing of the opening provided in the chemical pump housing.

8. The chemical pump assembly of claim 1 wherein the pull tab is a second pull tab with the chemical pump assembly including:
a first pull tab which extends from the inner chamber to ambient atmosphere through the opening; and
a removable layer connected to the first pull tab which shields the reactor from air in the inner chamber until the removable layer is removed.

9. The chemical pump assembly of claim 8 further including a packet in which the reactor is placed, and adhesive is provided on the removable layer on a side facing the packet, with exception of a small section at and end of the removable layer opposite from the opening.

10. The chemical pump assembly of claim 1 wherein the cover body moves into the opening toward the inner chamber to provide an indication that the inner chamber is under negative pressure when the cover body is covering the opening and the inner chamber is under negative pressure.

11. The chemical pump assembly of claim 1 wherein the chemical pump assembly is connectable to a dressing defining an enclosed volume so that the inner chamber is in fluid communication with the enclosed volume.

12. A method for operating a pump assembly of a negative pressure system, the method comprising:
providing a fluid connection between a chemical pump housing and a dressing placed over a tissue site, the chemical pump housing having an inner chamber and an opening fluidly connected to the inner chamber;
activating a reactor located within the inner chamber of the chemical pump housing to enable the reactor to react with a selected gas found in air so as to consume the selected gas when exposed to air in the inner chamber; and
after activating the reactor, covering the opening with a cover disposed over at least a portion of the chemical pump housing for sealing the opening to prevent ingress of air through the opening into the inner chamber, wherein covering the opening includes removing a removable liner disposed over an adhesive on an underside surface of a cover body by pulling a pull tab of the removable liner that extends laterally away for the chemical pump housing in a lateral direction across the cover body.

13. The method of claim 12 wherein covering the opening further includes affixing the underside surface of the cover body to the chemical pump housing after removing the removable liner by pushing the underside surface at which the removable liner was removed toward the chemical pump housing.

14. The method of claim 12 wherein the pull tab is a second pull tab and activating the reactor includes:
pulling a first pull tab, which extends from the inner chamber of the chemical pump housing to ambient through the opening in the chemical pump housing, to remove a removable layer to expose the reactor located within the inner chamber to air.

15. The method of claim 12 wherein the cover is a thin or metallized film having a show surface facing away from the chemical pump housing and the underside surface facing toward the chemical pump housing.

16. The method of claim 12 wherein the removable liner includes a first section having the pull tab integrally formed therewith and a second section, the first and second sections independently removable relative to one another.

17. The method of claim 16 wherein the second section is removed from the underside surface of a corresponding portion thereof that is affixed to the chemical pump housing at a location offset relative to the opening.

18. A cover for a chemical pump assembly useful for negative pressure therapy comprising:
a cover body having an underside surface that faces an associated chemical pump housing, the underside surface having an adhesive thereon for affixing the underside surface to the associated chemical pump housing, and
a removable liner disposed over the adhesive on the underside surface, the removable liner including a pull tab extending laterally away from the chemical pump housing.

19. The cover of claim 18 wherein the removable liner is a thin film that includes a first section and a second section, the first and second sections independently removable from the cover body relative to one another, and wherein the pull tab is formed integrally with the first section.

20. The cover of claim 19 wherein the first and second sections define a slit line extending laterally across the cover body at a location where the first and second sections meet one another.
